(19) United States
(12) Patent Application Publication
(10) Pub. No.: US 2010/0160791 A1
(43) Pub. Date: Jun. 24, 2010

(54) PORCINE BILIARY TRACT IMAGING

(75) Inventors: Hanli Liu, Arlington, TX (US); Edward Livingston, Dallas, TX (US); Bo Ping Wang, Arlington, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/600,981

(22) PCT Filed: May 21, 2008
(86) PCT No.: PCT/US08/64435

§ 371 Date: Mar. 2, 2010

Related U.S. Application Data
(60) Provisional application No. 60/939,257, filed on May 21, 2007.

(51) Int. Cl. A61B 6/00

(52) U.S. Cl. 600/476; 600/407

(57) ABSTRACT

The present invention includes apparatus and method to prevent surgical injury. The invention incorporates near infrared (NIR) spectroscopy, which capitalizes on near infrared light's ability to penetrate deeply into tissues and spectroscopic capability to discern tissue's chemical properties. The present invention further characterized the NIR optical properties of bile containing structures as a clinically useful probe.

### BACKGROUND OF THE INVENTION

Without limiting the scope of the invention, its background is described in connection with near infrared (NIR) spectroscopy, more particularly, apparatus and methods to prevent biliary tract injury during a surgery using in-vivo near infrared spectroscopy.

Approximately 400,000 cholecystectomies are performed annually in the United States. The most important complication of the operation is bile duct injury (BDI). Injury prevention relies mostly on an individual surgeon's skill. As of yet no technology has been introduced that enables surgeons to visualize the bile ducts while operating. Routine intraoperative ultrasonography has been proposed for delineating biliary anatomy; however, the images are not very clear and identifying the relevant anatomy is challenging. Even in relatively small trials of routine intraoperative ultrasonography, major bile duct injuries do occur, demonstrating that the technique currently in use falls short as an injury-prevention strategy. Intraoperative ultrasound has not been adopted by surgeons for identifying portal anatomy during cholecystectomy. Currently, some optical techniques do exist for characterizing tissue structures and chemical properties.

For example, U.S. Pat. No. 5,807,261 teaches a tool for nondestructive interrogation of the tissue including a light source emitter and detector, which may be mounted directly on the surgical tool in a tissue contacting surface for interrogation or mounted remotely and guided to the surgical field with fiber optic cables. The light source may be broadband and wavelength differentiation can be accomplished at the detector via filters or gratings, or using time, frequency, or space resolved methods. Alternatively, discrete monochromatic light sources may be provided which are subsequently multiplexed into a single detector by time or by frequency multiplexing. The optical sensing elements can be built into a surgical tool end effector tip such as a tissue grasping tool which has cooperating jaws (bivalve or multi-element). In the '261 patent, the light source (or the fiber optic guide) is mounted on one jaw and the detector (or fiber optic guide) is mounted in the opposing jaw so that the light emitter and detector are facing one another either directly (i.e., on the same optical axis when the tool is closed) or acutely (i.e., with intersecting optical axes so that the light emitted is detected), and the sensor works in a transmission modality.

Arrangements with the optical components mounted on the same member of a single member or a multi member structure, operating in a reflective modality, are disclosed.

Another example can be found in U.S. Pat. No. 5,711,755. The '755 patent teaches endodiagnostic apparatus and methods by which infrared emissions within the range including 2 to 14 micrometers may be visualized in the form of encoded images to permit differential analysis. The endoscopic apparatus includes a refractive objective lens for forming a real image of interior structures of interest, a relay system consisting solely of refracting elements for transferring the real image to an intermediate image plane conjugate to the objective image plane, and a refracting coupling lens for forming a final image of the intermediate image in a detector plane in which an IR detector sensitive in the range including 2 to 14 micrometers may be placed near the proximal end of the apparatus.

Yet another example can be found in U.S. Pat. No. 5,944,653. The '653 patent discloses dual channel endodiagnostic apparatus and methods by which infrared emissions within the range including 2 to 14 micrometers may be visualized in the form of encoded images to permit differential analysis. The endoscopic apparatus has an IR channel and a visible channel. The IR channel includes a refractive objective lens for forming a real image of an interior structure of interest, a relay system consisting solely of refracting elements for transferring the real image to an intermediate image plane conjugate to the objective image plane, and a refracting coupling lens for forming a final image of the intermediate image in a detector plane in which an IR detector sensitive in the range including 2 to 14 micrometers may be placed near the proximal end of the apparatus. The IR and visible channels are arranged to visualize substantially the same subject matter.

U.S. Pat. No. 7,236,815 introduces fluorescence spectral data acquired from tissues in vivo or in vitro that is processed in accordance with a multivariate statistical method to achieve the ability to probabilistically classify tissue in a diagnostically useful manner, such as by histopathological classification. The apparatus includes a controllable illumination device for emitting electromagnetic radiation selected to cause tissue to produce a fluorescence intensity spectrum. Also included are an optical system for applying the plurality of radiation wavelengths to a tissue sample, and a fluorescence intensity spectrum-detecting device for detecting an intensity of fluorescence spectra emitted by the sample as a result of illumination by the controllable illumination device. The system also includes a data processor, connected to the detecting device, for analyzing detected fluorescence spectra to calculate a probability that the sample belongs in a particular classification. The data processor analyzes the detected fluorescence spectra using a multivariate statistical method. The five primary steps involved in the multivariate statistical method are (i) preprocessing of spectral data from each patient to account for inter-patient variation, (ii) partitioning of the preprocessed spectral data from all patients into calibration and prediction sets, (iii) dimension reduction of the preprocessed spectra in the calibration set using principal component analysis, (iv) selection of the diagnostically most useful principal components using a two-sided unpaired student's t-test and (v) development of an optimal classification scheme based on logistic discrimination using the diagnostically useful principal component scores of the calibration set as inputs.

Finally, United States Patent Application Publication Number 2006/0247514 teaches a method for irradiating a biological sample with far infrared (FIR) irradiation, including providing tunable FIR irradiation, removing X-rays from the irradiation, and irradiating at least one biological sample with the tunable FIR irradiation, wherein at least a component of the biological sample undergoes at least one of a conformational change or a phase change in response to the irradiating. An FIR irradiation device is disclosed, including an FIR source producing an FIR irradiation having a tunable wavelength, the source being capable of continuous-wave output, and a filter receiving the irradiation from the source.

The present inventors recognized that none of the above reference characterizes optical properties of bile containing structures as a clinically useful probe and there is a need for devices that can eliminate BDI.

### SUMMARY OF THE INVENTION

The present invention provides NIR spectroscopy combined with visible light spectroscopy to determine the spectroscopic properties of the biliary tree and its adjacent structures. Reflectance measurements using a fiber probe were obtained. Radial Basis Functions (RBF) were used to characterize the reflected light spectra. Parameters describing the RBF were then used to classify tissues based on their observed spectra using machine automation.

In one aspect, the present invention is an apparatus to visualizes one or more tissues in vivo. The apparatus has an electromagnetic radiation source capable of producing a continuous wave broadband light, at least one optical probe connected to the electromagnetic radiation source, a multi-channel CCD array spectrometer detector connected to the optical probe capable of collecting near infrared wavelength emissions, at least one computer connected to the multi-channel CCD array spectrometer detector. The computer includes one or more image evaluation algorithms, and at least one display connected to the computer to generate images from the one or more tissues and display results from the image evaluation algorithms. The optical probe is typically a fiber optical bundle having one or more non-bifurcated channels for light delivery fibers and one or more bifurcated channels to collect the near infrared wavelength emissions. The near infrared wavelengths emissions used has wavelength between about 550 nm and about 900 nm. In one aspect, the present invention examines tissues such as biliary tree of an animal or human. In another aspect, the present inventions uses one or more image evaluation algorithms such as a Radial Basis Function algorithm to facilitate the characterization of the one or more tissue based on observed reflectance spectra and to distinguish between tissue structures, a Minimal Distance Method algorithm to classify the one or more tissues, a two-layer diffusion model algorithm to localize heterogeneities, and/or a linearized image reconstruction algorithm to obtain ultrasound-like, two-dimensional images.

Yet in another aspect, the present invention demonstrates methods of imaging one or more tissues (e.g. a biliary tree) in an animal or a human subject during a surgical operation. The method typically includes directing a continuous wave broadband light towards the one or more tissues using at least one optical probe, collecting near infrared wavelength emissions from the one or more tissues using the optical probe, analyzing the near infrared wavelength emissions using one or more image evaluation algorithms, and/or reconstructing a two-dimensional or three-dimensional optical map of the one or more tissues based on results derived from the one or more image evaluation algorithms. The tissue examined using the present invention can use a Radial Basis Function algorithm to facilitate tissue identification based on observed reflectance spectra and to distinguish between tissue structures or use a Minimal Distance Method algorithm to classify the tissues. In one aspect, the Minimal Distance Method uses the equation:

$$S ( λ ) = {\sum }_{i = 1}^{N} {a}_{i} {e}^{( \frac{- {( λ - {λ}_{i} )}^{2}}{2 {σ}_{i}^{2}} )}$$

In some aspects, the one or more image evaluation algorithms also includes a two-layer diffusion model to localize heterogeneities, or a linearized image reconstruction algorithm to obtain ultrasound-like, two-dimensional images.

### BRIEF DESCRIPTION OF THE DRAWINGS

For a more complete understanding of the features and advantages of the present invention, reference is now made to the detailed description of the invention along with the accompanying figures and in which:

FIG. 1(a) is a magnified view of the hand-held, multi-channel NIR probe tip;

FIG. 1(b) is a cross-sectional schematic of the probe with the fiber diameter and separation dimensions;

FIG. 1(c) is a photograph of the NIR optical system and multi-channel CCD spectrometer;

FIG. 2 is a normalized reflectance spectra obtained from porcine biliary tissues and blood vessels;

FIG. 3 is a spectra showing two classes of observed gallbladder spectra;

FIG. 4(a) is a spectra from results obtained from fitting of the portal vein using RBF;

FIG. 4(b) is a spectra from results obtained from fitting of the hepatic artery using RBF;

FIG. 5 is a plot of the A, B and C coordinates for artery, vein and gallbladder following RBF fitting of the observed spectra;

FIG. 6 is a graph of classification results from the testing phase of the simulation demonstration;

FIG. 7 is a plot of fitting observed spectra with radial basis functions. λᵢ, aᵢ, and σᵢ for i=1, 2, 3 are the fitted parameters describing the Gaussians curves used to fit the reflectance spectrum obtained from an artery;

FIG. 8 is a plot of simulated changes in reflectance due to a hidden object (5×5 mm2 in size) at depth of Z below the surface;

FIG. 9(a) is a schematic diagram showing Monte Carlo simulated photon visit probability profile in fat with a source-detector separation of 7 mm and with an artery cross-section superimposed,

FIG. 9(b) is a schematic diagram showing variable “banana” patterns as a function of source-detector separations;

FIG. 9(c) is a schematic diagram showing how a moving banana-shaped probe volume intersects the underlying absorbing objects, such as an artery;

FIG. 9(d) is a schematic diagram showing how a moving banana-shaped probe volume intersects the underlying absorbing objects, such as an artery and a bile duct;

FIG. 10(a) is a schematic diagram of photos of the 1st fiber-optic;

FIGS. 10(b) and 10(c) are schematic diagrams of a multi-channel probe;

FIGS. 11(a) and 11(b) are pictures of the 2nd scanning probe with 7 channels of fiber bundles that was connected to the light sources and multi-channel spectrometer;

FIG. 11(c) is a picture of a close view of the 7-channel fiber-optic imaging probe with 3 channels being bifurcated for both light delivery and detection;

FIG. 12(a) is a picture of the instrument setup for a simulated biliary tract object embedded in intra-lipid solution.

FIG. 12(b) is a schematic of the scanned geometry for the hidden object (8 mm in diameter).

FIG. 12(c) is a picture showing three reconstructed images of the hidden object. The Y-axis represents distance.

FIG. 13 is a plot of reflectance taken from a 3-mm (blue) and 5-mm (pink) object.

FIG. 14(a) is an image of comparison between the original and corrected reflectance images for a 5-mm-diameter object

FIG. 14(b) is an image of comparison between the original and corrected reflectance images for two 3-mm-diameter objects, both hidden 4-mm below the surface of 1% intralipid solution; the S-D separation was 6 mm for case 14(a) and 3 mm for case 14(b). The corrected images were obtained using spatial 2nd derivative image processing.

FIG. 14(c) is a plot of the reflectance profile for case 14(b) and clearly shows improved spatial resolution from the processed data.

FIG. 15 (a) is a schematic diagram of a phantom setup for testing a multi source-detector, linear-array imaging probe with a 3-mm object imbedded 2 mm-4 mm below the probe surface.

FIG. 15 (b) is a schematic diagram showing top view of the interface between the linear-array optical probe and the phantom containing a hidden absorber.

FIG. 16(a) is a picture demonstration of an extended linear-array setup by scanning the array probe back-to-back (blue and pink bar)

FIG. 16(b) is a picture demonstration combining the two sets of reflectance readings together. The extended array provides the accurate location of the hidden absorbing object, which was near the edge of the original array probe;

FIG. 17 (a) is a graph reconstructed apparent absorption coefficients, obtained by fitting the semi-infinite medium model, for (a) an embedded 2-mm diameter artery that is 4 mm centered below the fatty surface;

FIGS. 17 (b) and (c) are plots showing the reconstructed apparent absorption coefficients for the same artery 7 mm away from a 4 mm diameter bile duct when the source is near the true locations of (b) the artery and (c) bile duct, respectively. Also notice that the blue and red curves shown in (b) and (c) are switched in magnitude, illustrating that artery at 866 nm has more absorption whereas bile duct has more absorption at 716 nm. Such spectrally dependent data analysis permits locating multiple absorption heterogeneities simultaneously;

FIG. 18 is a picture showing the capacity of this approach to localize the artery and the bile duct when their spatial separation is variable, as is often the case in a real surgery scenario;

FIG. 19 (a) is a diagram showing ideal or actual absorption perturbation map where most pixels have the uniform absorption coefficient of fat and one pixel has that of arterial blood;

FIG. 19 (b) is a plot of a reconstructed absorption perturbation map using accurate background scattering coefficient of fat;

FIG. 19(c) is a reconstructed absorption perturbation map using inaccurate background scattering coefficient of fat. In both (b) and (c) cases, the hidden absorption object can be reconstructed with good accuracy for its location or depth;

FIG. 20(a) is a picture showing ideal or actual absorption perturbation map where most pixels have the uniform absorption coefficient of fat and 2×2 pixels have uniform heterogeneity.

FIG. 20(b) is a picture showing a reconstructed absorption perturbation map using accurate background scattering coefficient of fat;

FIG. 20(c) is a picture of reconstructed absorption perturbation map using inaccurate background scattering coefficient of fat. In both (b) and (c) cases, the hidden absorption object give more weight to the pixels closer to the tissue surface;

FIG. 21 is a plot of comparison between the fitted (red) and demonstration data (blue).

FIG. 22(a) is a schematic diagram to show the computer-simulated measurement domain;

FIG. 22(b) is a schematic diagram showing discrete locations of the measurements on the surface;

FIG. 22(c) is a diagram of a 2-D reconstructed vessel or biliary tree locations;

FIG. 22(d) is a smoothed 3-D image map;

FIG. 23 is a picture of overall system design and principles for a multi-spectral, multi-separation, visible-to-NIR imaging system;

FIG. 24(a) is a picture of the oval-shaped cross-section of the probe;

FIG. 24(a) is a schematic diagram of design of the intra-operative NIR probe. This design and development corresponds to component 1 shown in FIG. 21;

FIG. 25 is a picture of an existing 8-channel, tomographic imager;

FIG. 26 is a picture of the NIR multi-channel imaging system. This design and development corresponds to component 2 shown in FIG. 21; and

FIG. 27 is a diagram of a probe laterally over the tissue surface.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Development of gallstones is very common and these cause pain or infection. Consequently, there are more than 400,000 operations to remove the gallbladder (cholecystectomy) in the United States annually. Below the gallbladder is a duct that carries bile from the liver to the intestines and is close to the gallbladder. This duct is buried under fatty tissues and cannot be visualized by the operating surgeon. Although only occurring in about 0.5% of cases, Bile Duct Injury (BDI) can occur and when it does, have devastating consequence. In an era where surgeon-induced injury is not acceptable, a pressing need exists for developing technologies to minimize or eliminate the risk for these injuries by providing a way for surgeons to locate these ducts during surgery. Currently, only a couple of methodologies are available for intraoperative bile duct (BD) imaging. For example, intraoperative ultrasound has been advocated but has never proven to be useful clinically. Since its introduction by Mirizzi in 1937, the current standard is intraoperative cholangiography (IOC). IOC requires identification and cannulation of the cystic duct that connects the gallbladder to the bile duct. Dye is typically injected and radiographs obtained. At best, IOC can reduce but not eliminate the risk of BDI. IOC can itself result in injury since it requires dissection and cannulation of the cystic duct.

In one aspect, the present invention demonstrates new technologies that can replace IOC. One goal is to develop imaging devices enabling surgeons to visualize critical structures adjacent to the gallbladder without first needing to dissect any tissues. The present invention relies on hyperspectral imaging of the porta, facilitating chemometric (assessment of chemical properties of tissues) and identification of tissues using a video-based device. As light travels through tissues, it is scattered, reducing the resolving power of any imaging device and renders the data unusable. For this reason, the present inventors developed a novel imaging system based on an optical probe having discrete channels to collect reflected visible-to-near infrared (NIR) light. Reflected light spectra are analyzed for wave patterns characteristic of the unique chemical composition of a tissue facilitating its identification. The probe approach facilitates structure identification in the presence of light scattering and enables modeling of the gastroduodenal ligament's contents' spectroscopic characteristics. This information allows the development of video imaging devices compatible with laparoscopic systems enabling surgeons to see the BD while operating and, eradicating BDI.

Light traveling through tissues is absorbed and scattered by blood, sub-cellular structures and the extracellular matrix. Optical radiation at near infrared (NIR) wavelengths (600-900 nm) penetrates superficial tissues more deeply than at visible (vis) ones (400-600 nm) because oxygenated (HbO) and deoxygenated (Hb) hemoglobin in blood absorb light less strongly in the NIR spectral window. The low absorption and high scattering of NIR light traveling through tissues has spurred the development of diffuse tomographic imaging. These methodologies are in essence efforts to compensate for the strong blurring of images due to the multiple scattering of NIR light and aim to reconstruct the true spatial distribution of Hb and HbO in tissues. Both static and functional maps of blood absorption can thus be made to depths of 1-3 cm below the measurement surface. Furthermore, optical spectroscopy of tissues can provide spectral fingerprints of tissue types and can thus be used to differentiate between them in vitro as well as in vivo.

The present invention shows that the spectroscopic characteristics of fat, blood and bile are sufficiently different such that good contrast can be obtained between the various biliary structures with visible to near infrared imaging. The capacity to differentiate between the spectral reflectance characteristics of these tissue types enabled the present inventors to develop a novel, laparoscopic-capable spectroscopic imaging system that visualizes the biliary tree during cholecystectomy. Development of such a system reduces surgical time and minimizes the risk of complications and expense attributable to intraoperative cholangiography.

The present invention can discriminate between bile and blood containing structures. Demonstrations were made by measuring NIR reflected light spectra from the gallbladder, arteries and veins using animal models in vivo. A novel classification algorithm that identifies the imaged tissues from the observed spectra was also developed. To examine the classification accuracy, classification method was used for the small sample size (n=8), followed by the classification procedures using a large computer-simulated data set showing an improved sensitivity and specificity. This enable the construction of a probe useful for intraoperative biliary tree imaging of the structures contained within the porta hepatis. As such, the present invention eliminates bile duct injury during cholecystectomy.

Pigs (n=8) weighing 75 kg were fasted for 12-16 hours before surgery. Because NIR reflected spectra are influenced by the oxygenated state of flowing blood, these demonstrations were performed in living animals in order to mimic human, intraoperative situation. Isofluorine general endotracheal anesthesia was administered, the pigs placed in a supine position and the abdomen opened. The gallbladder was identified and used to orient the anatomy such that the BD, hepatic artery and portal vein are identified.

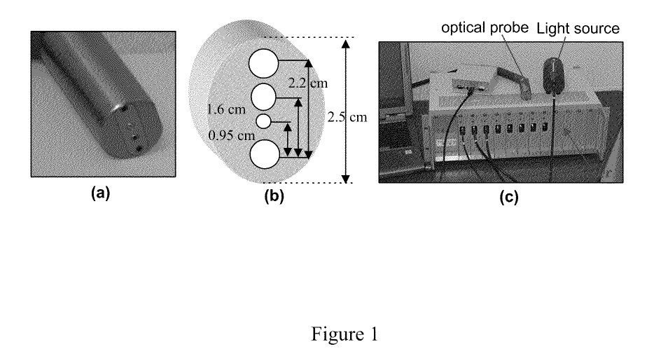

Measurements were obtained from the tissues containing the bile ducts and associated structures of live (n=8) pigs using a spectroscopic system consisting of a hand-held probe, a multi-channel spectrometer and a laptop computer (FIG. 1). FIG. 1(a) shows magnified view of the hand-held, multi-channel NIR probe tip. FIG. 1(b) is a cross-sectional schematic of the probe with the fiber diameter and separation dimensions. The solid circle presents the light-delivery fiber and the open circles are for the fibers to carry the reflected light to the spectrometer. FIG. 1(c) is a photograph of the NIR optical system and multi-channel CCD spectrometer. The multi-channel spectroscopic probe had several different distances between the light source and the detector fibers. Distances between the source and detector fibers determine the tissue depth the probe images such that shorter distances image structures that are relatively shallow in the tissue and larger separations image more deeply. Current demonstrations show that for porcine gastrohepatic ligament structures the optimal source-detector separation was 0.95 cm.

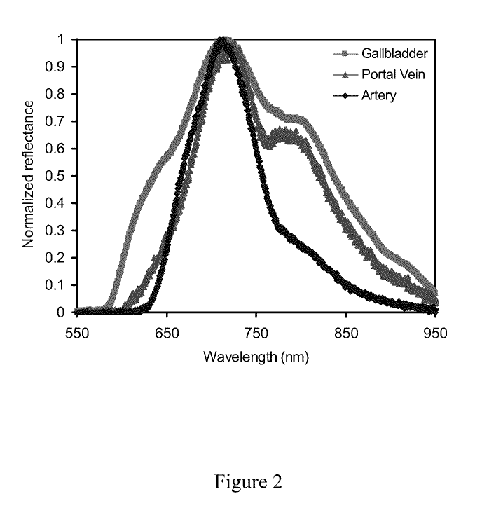

Light from a tungsten-halogen lamp with continuous wave broadband light was delivered through the source fiber onto the tissue. Reflected light from the tissue is captured by the detector fiber and is channeled to a multi-channel CCD array spectrometer. Reflectance spectra ranging from 550-900 nm were obtained of porcine biliary tissues and blood vessels from 8 anesthetized pigs. Each tissue yielded a unique spectrum (FIG. 2). FIG. 2 is a normalized reflectance spectra obtained from porcine biliary tissues and blood vessels. Graphs were normalized to the highest peak value to facilitate comparisons of the spectra from differing tissues. The top curve represents the observed spectra for the gallbladder, the middle curve for the portal vein and the lower curve for arteries.

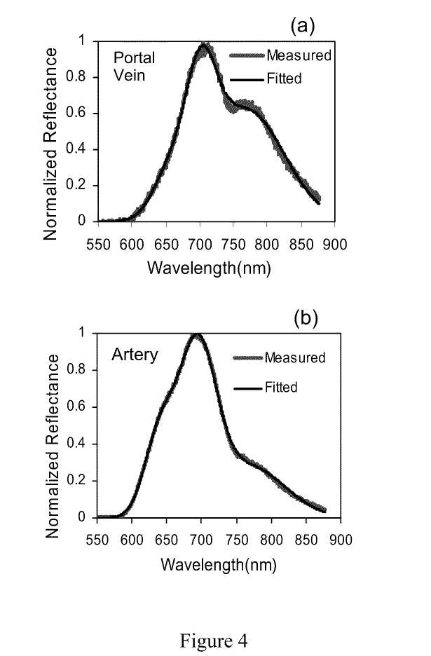

For each tissue imaged, Radial Basis Functions (RBF) were used to characterize the reflected light spectra. The 9 parameters characteristic for each spectrum were determined. The ability of these 9 parameters to reconstruct the observed spectra is demonstrated in FIGS. 4(a) and 4(b). FIG. 4 illustrates results obtained from fitting the spectra of the (a) portal vein and (b) hepatic artery using RBF. The red curves represent the observed spectra and the black lines the reconstructed spectra derived from the 9 parameters of the RBF fit. (R²>0.99). The mean, standard deviation (s.d.) and coefficient of variation (c.v.) were calculated for each of the 9 parameters for all tissues (gallbladder, hepatic artery and portal vein) (Table 1). Table 1 shows mean, standard deviation and coefficient of variation for each of the nine fitted parameters of the RBF for all three tissue types measured. The greatest heterogeneity was found for a₁, a₂, a₃, σ₂ and σ₃, demonstrating that they are the most informative in terms of discriminating different tissue types.

TABLE 1

| Organs | a₁ | a₂ | a₃ | λ₁ | λ₂ | λ₃ | σ₁ | σ₂ | σ₃ |
|---|---|---|---|---|---|---|---|---|---|
| Artery | 0.18 | 0.96 | 0.07 | 777.39 | 683.28 | 608.19 | 46.27 | 34.59 | 11.79 |
| Gallbladder | 0.67 | 0.67 | 0.35 | 768.89 | 687.53 | 622.34 | 63.33 | 32.84 | 21.32 |
| Portal vein | 0.63 | 0.56 | 0.24 | 763.02 | 699.94 | 657.59 | 57.91 | 21.72 | 26.72 |
| Mean | 0.49 | 0.73 | 0.22 | 769.77 | 690.25 | 629.37 | 53.50 | 29.72 | 19.94 |
| s.d. | 0.27 | 0.21 | 0.14 | 7.23 | 8.66 | 25.44 | 8.62 | 6.98 | 7.56 |
| c.v.* | 55.15 | 28.31 | 64.12 | 0.94 | 1.25 | 4.04 | 16.11 | 23.49 | 37.91 |

Parameters with greater heterogeneity are more informative and can potentially be useful for discriminating between tissue types. The center of the Gaussians was constant between tissues, thus, these have little potential for differentiating tissue types. The greatest heterogeneity was in the height parameter, a, followed by the width parameter a. A coefficient of variation exceeding 20% has sufficient heterogeneity to be useful for tissue identification.

The greatest heterogeneity was observed in a₁, a₂, a₃, σ₂ and σ₃. Reduction of the number of working variables was accomplished by creating combination variables defined as:

A=(aᵢ/a₂), B=(a₃/a₂) and C=(σ₂/σ₃). The 3 parameters A, B and C fully characterized the observed optical profiles and could be used to classify spectra observed from unknown tissues into their appropriate tissue type category.

Using a probe to identify the structures contained in the gastroduodenal ligament requires linking measured spectra to those characteristics for the tissue of interest. To accomplish this, the observed spectra must be characterized. This is accomplished by the RBF analysis described above. Parameters A, B and C should be unique for each type of tissue. Once subjected to a classification algorithm, these parameters can be used for identifying tissue types.

### Algorithm Development for Tissue Classification

Minimal Distance Method (MDM) was applied to classify observed spectra into the correct tissue type. MDM is a statistical matching process commonly used in pattern recognition for remote sensing and image processing. The parameters a₁, a₂, a₃, σ₂ and σ₃ are used to represent a simulated spectrum, Sᵢ(λ). Simulations were performed by creating a library of 900 randomly generated a₁, a₂, a₃, σ₂ and σ₃ values that had the same mean and standard deviation as the measured a₁, a₂, a₃, σ₂ and σ₃ values obtained in the 8 pigs. From this library, 900 spectra were created by randomly selecting a₁, a₂, a₃, σ₂ and σ₃ combinations from the simulated library. Nine hundred simulated spectra were created each for the gallbladder, arterial and venous spectra totaling 2,700 simulated spectra.

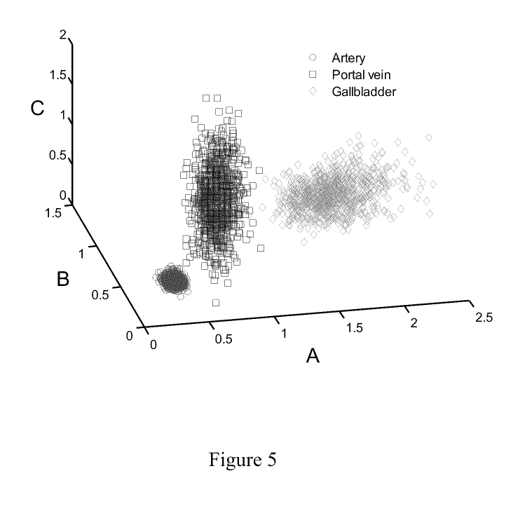

For the initial phase of the classification algorithm, the a₁, a₂, a₃, σ₂ and σ₃ parameters were combined for each tissue to derive 700 estimates of the parameters A, B and C. These are represented as “clouds” in FIG. 5. FIG. 5 is a plot of the A, B and C coordinates for artery, vein and gallbladder following RBF fitting of the observed spectra. Each point represents a simulated A, B and C dataset derived from a random set of a₁, a₂, a₃, σ₂ and σ₃ parameters derived in the simulation studies. The randomly generated parameters had the same mean and standard deviation values as did those obtained from actual pig measurements. Each cloud contains 700 simulated data points. Each colored cloud represents modeled artery, vein, and gallbladder. The centers of the clouds in the A-B-C space correspond to the mean locations of the 700 data points for each tissue type. It is seen that the three data clouds are well separated with either small (for artery) or large (for Gallbladder) extended boundaries. Such boundaries can be approximately expressed by standard deviations of the distances that are between the individual cloud points and the respective cloud centers, σ(j), where j runs for three types of tissues: artery, portal vein, and gallbladder. Any data point in the A-B-C space within a particular “cloud” were classified to the corresponding type of tissue. The mean and standard deviation for each tissue cloud was calculated.

Tissue assignment follows minimization of the Mahalanobis distance. Euclidian distances are often calculated to determine distances between points in space. In order to account for complex shapes of three dimensional point distributions and scaling phenomenon, a more complex calculation is necessary. The Mahalanobis distance is used in automated feature identification algorithms and is frequently used to address the complexities of measuring distances in space. The Mahalanobis distance is determined from the mean, variance and covariance of data points in the A-B-C space and is defined as D_{N}(i,j)=D(i,j)/σ(j), where D(i,j) is the distance calculated from the ith data point to the jth center of its corresponding tissue group (or its cloud in FIG. 5), σ(j) is the s.d. that was calculated for the jth tissue from the training algorithm, and i=1, . . . 200 for multiple data points; j=1, 2, 3 for artery, portal vein, and gallbladder. This has the advantage of not being dependent on the measurement scale (scale-invariant) and avoids problems that arise with calculating Euclidian distances when data are highly correlated. An unknown tissue may have its A, B and C parameters calculated and the Mahalanobis distance between these coordinates and the centers for each of the tissue clouds calculated. The smallest distance between one of the tissues and the unknown was used to classify the unknown as being of that tissue type.

The remaining 200 A, B and C points were used from the simulated data set to demonstrate the use of the tissue classification algorithm. The three normalized distances D_{N}(i,j)=D(i,j)/σ(j) between a simulated data point that is associated with a tissue type and each of the cloud centers were computed where σ(j) is the standard deviation for the cloud associated with a certain tissue type. The calculated minimum distance between a tissue cloud center and the unknown point is used to associate the unknown with that particular tissue. Each of the 200 simulated A, B and C parameters were derived a data set with characteristic values for a particular tissue. The number of times these tissues were identified during the classification demonstration.

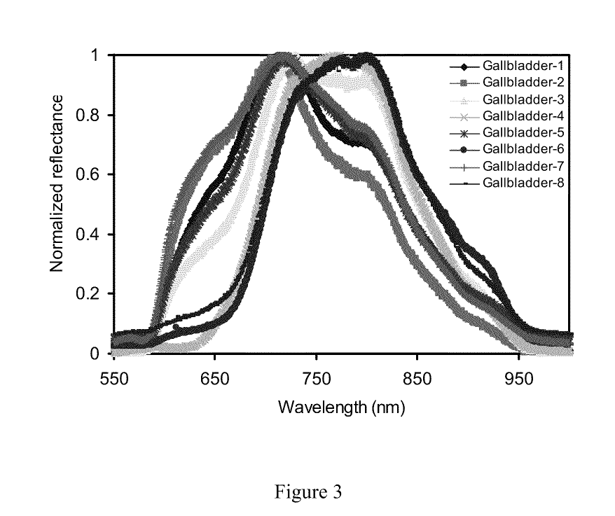

Averaged reflectance spectra were taken from the gallbladder, hepatic artery and portal vein for all eight pigs (FIG. 2). These spectra all have peaks at approximately the same wavelengths but differ in amplitude and width. For analytic purposes, the spectra were normalized such that the amplitude ranged from 0.0 to 1.0. Spectra for bile containing structures, oxygenated and deoxygenated blood should have unique waveforms. Because of its large size, the gallbladder measurements provided the best spectra representative for bile and bile containing structures. However, there were 2 distinct types of gallbladder spectra observed (FIG. 3).

FIG. 3 shows two classes of observed gallbladder spectra. The chemical nature underlying the two spectra was not discerned. Each spectrum was obtained from one of the 8 pigs. These most likely represent differing bile compositions. Although there were two patterns of gallbladder spectra, they were all used for calculation of the RBF coefficients. RBF's were determined for the tissue types was imaged and averaged from all the animals, as presented in Table 1. Each of 9 RBF coefficients are further presented as the mean and standard deviation over the three structures of tissues. Coefficients of variation for each coefficient are presented and were found to exceed 20% for a₁, a₂, a₃, σ₂ and σ₃. The overall mean values were determined from the ratios A=(aᵢ/a₂), B=(a₃/a₂) and C=(σ₂/σ₃). They were: artery: A=0.19, B=−0.07, C=2.93; vein: A=1.13, B=0.43, C=0.81; gallbladder: A=1.0; B=0.52, C=1.54. The ability of the A, B and C parameters to reconstruct the original observed spectra is demonstrated in FIG. 4.

The newly developed classification algorithm facilitates tissue identification based on observed reflectance spectra. As described above, observed spectra were processed and clearly distinguish between the three structures imaged: artery, vein, and gallbladder. These three types of tissues have distinct spectral features and are of reasonable sizes relative to the probe's dimensions for the measurements, resulting in minimal signal interference from background structures. As seen in FIG. 5, the centers of the three data clouds in the 3-dimensional space are well separated. RBF coefficient calculation for an unknown tissue enables a surgeon to classify a tissue as arterial, venous or bile containing when the measured coefficients are mapped to the 3D A-B-C space.

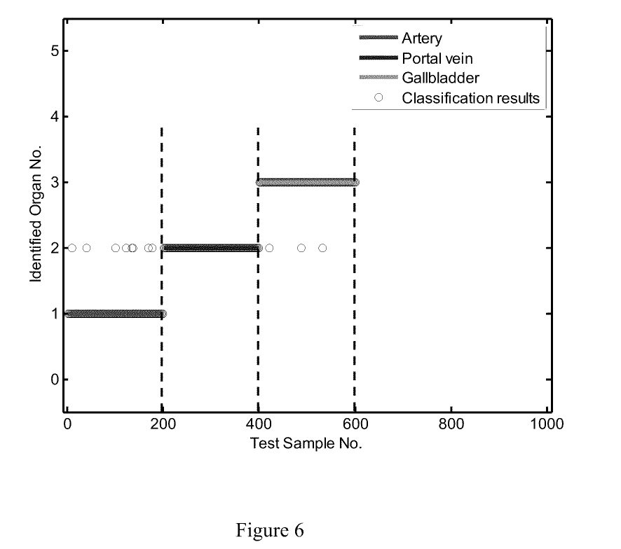

FIG. 6 shows the 200 simulated datasets for each tissue type used for the classification algorithm. Each tissue had 200 randomly generated data points that had the same mean and standard deviation as the measured A, B and C values. The MDM algorithm correctly classified all venous points and all but 3 points for the gallbladder and 8 for the artery. Given the wide separation of the point “clouds” in the A, B and C parameter space, the ability to correctly classify unknown tissue into gallbladder, artery or vein based on the A, B and C values calculated from RBF fitting of observed spectra is very good.

The respective sensitivity and specificity for tissue classification were excellent as shown in Table 2.

TABLE 2

| Tissue | Sensitivity | Specificity |
|---|---|---|
| Artery | 96% | 100% |
| Portal vein | 100% | 100% |
| Gallbladder | 98.5% | 98.2% |

### Radial Basis Functions (RBF)

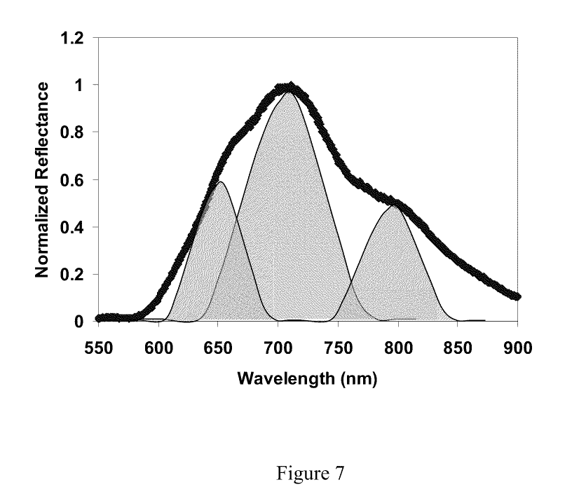

Functions are mathematical expressions that describe data transformation. They are equations that describe an observed spectral waveform. Basis functions are those that in linear combination can describe a waveform. In FIG. 7, an observed optical spectrum can be recreated by combining the 3 Gaussian shaped curves shown below it. A radial function has a center and can be described entirely in terms of its distance, i.e. its radius, from the center. Such functions are radially symmetric. RBF are demonstrated in FIG. 7. Equation 1 describes a typical Gaussian, bell-shaped response, S(λ), that depends on the distance between a reference point, λ, located somewhere along the waveform, and the center of the Gaussian curve, λᵢ. The Gaussian curve's width is characterized by σᵢ and its amplitude by aᵢ.

$$S ( λ ) = {\sum }_{i = 1}^{N} {a}_{i} {e}^{( \frac{- {( λ - {λ}_{i} )}^{2}}{2 {σ}_{i}^{2}} )} \qquad ( 1 )$$

The σᵢ parameter in Equation 1 controls the RBF's shape and is called a local dilation parameter or a shape parameter. It has been found that reflected light spectra observed can be fit by 3 Gaussian curves in the RBF (i.e., N=3). Each of these curves can be described by 3 parameters: the center point of the Gaussian λᵢ, the shape parameter σᵢ and the amplitude factor aᵢ.

BDI remains an important clinical problem. As of yet, no technology has been developed that has the potential for eliminating this important surgical complication. Routine intraoperative cholangiography (IOC) has been advocated as a risk reduction strategy; but, at best, this can somewhat reduce but not eliminate the complication. Even the modest reductions in BDI associated with routine IOC come at a high cost and population-based data suggests that few hospitals have adopted a routine IOC policy. Intraoperative ultrasound has been proposed for intraoperative bile duct imaging but has not proven to be very effective nor widely adopted in clinical practice.

The present invention uses an NIR fiberoptic probe approach for intraoperative bile duct identification. This system capitalizes on the tissue penetrating properties of NIR light. It also uses spectral information so that tissues can be identified by their chemical composition.

The present invention demonstrates that the tissue identification algorithm disclosed herein is robust. This means that the parameters selected (A, B and C) were sufficiently unique that tissues could be classified with little chance of error. It also shows that bile, arterial blood and venous blood were widely separated in the A, B, C parameter space (FIG. 4) and that the classification algorithm performed well.

### Design and Development of a Laparoscopic Optic Probe

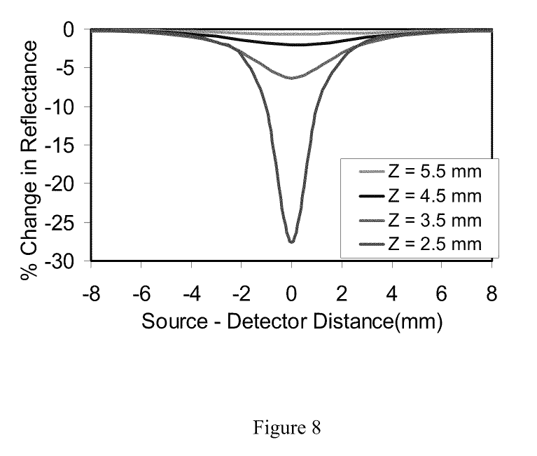

Perturbations in Optical Reflectance Signal from Absorbing Heterogeneities. In order to design an optimal spectroscopic probe, the present invention used Monte Carlo (MC) simulation of optical reflectance signals transmitted through a scattering medium embedded with a 5.5 mm² high-absorbing object having similar optical properties to venous blood, simulating the portal vein. The scattering medium had fat-like optical properties. The object was placed at variable depths (Z) and the source-detector separations were varied from 0 to 8 mm. The percentage change in reflectance due to the absorber was recorded. FIG. 8 demonstrates the MC simulations for percentage changes in reflectance at 716 nm with various depths.

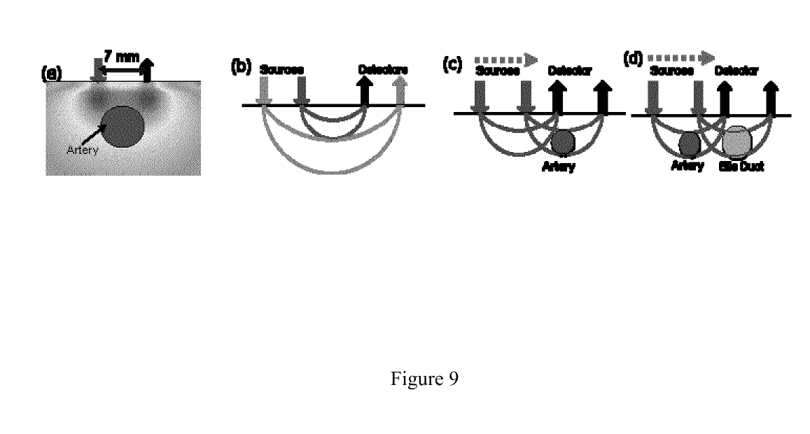

The reflectance change observed when a measurement probe is on top of an absorbing heterogeneity (blood vessel, bile duct) is directly related to the depth selectivity of the probe. The number of photons of a given vis-to-NIR wavelength that reach a certain tissue depth is a function of source-detector separation and of the tissue optical properties. Due to multiple photon scattering, the tissue volume that photons visit, known as the photon measurement density function, takes a shape that is popularly referred to as a “banana” (FIG. 9(a), Monte Carlo simulated photon visit probability profile in fat with an artery cross-section superimposed). Each source-detector pair defines its own banana-shaped photon visit distribution; the greater their separation, the deeper is the mean tissue depth that is visited (FIG. 9(b)), albeit by fewer photons due to tissue absorption and scattering. Here, data from different source-detector separation combinations were analyzed, while sequentially moving or switching the sources along the tissue surface from left to right (FIG. 9(c), blue arrow indicates source displacement in sequential reflectance measurements—the displacement of only one source-detector pair is shown for clarity).

Moving the source results in movement of the banana-shaped probe volume along the same direction. As the latter is moved from left to right, it intersects underlying absorbing heterogeneities (FIG. 9(c)—artery; FIG. 9(d)—artery and bile duct), which results in a detected signal change at the surface (as in FIG. 8). When the tissue probe volume intersects an absorbing heterogeneity, the detected reflectance changes are used to localize that heterogeneity both on the tissue surface and depth-wise.

The top side of biliary tree structures is typically found at 2-6 mm depths from the fatty tissue's surface. To emulate this real-life tissue geometry, Monte Carlo spectrally-resolved reflectance measurements were simulated (with different wavelengths) for biliary tree structures centered at a depth of 4 mm (artery: 2 mm diameter; bile duct: 4 mm diameter). To target those depths and beyond, the depth-resolved visit probability for vis-to-NIR photons for different source-detector separations and mammalian fat optical properties were plotted. For example, FIG. 9(a) shows the color-coded photon visit probability at 716 nm for a source-detector separation of 7 mm. This shows that the heterogeneity localization algorithms include reflectance data in the 3.5-14 mm source-detector separation range.

### Probe Design and Implementation

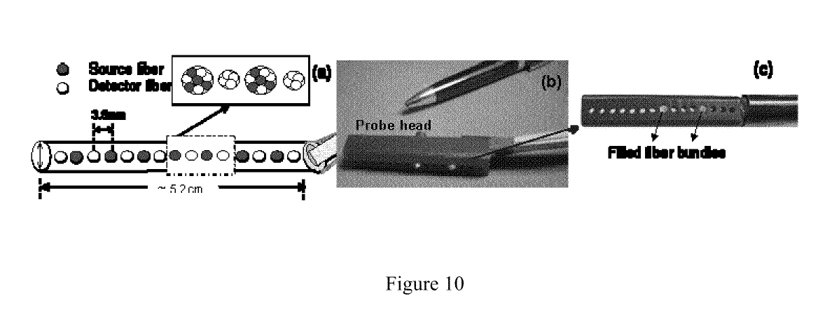

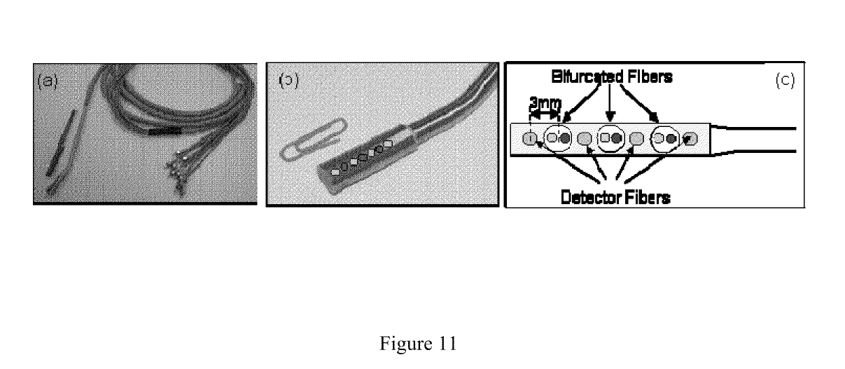

The schematic design for the probe is demonstrated in FIG. 10(a). FIGS. 10(b) and 10(c) show the fiber-optic probe according to the design. This device has two of the channels filled with fiber bundles. A 2^{nd} device of the fiber-optic probes with 7 channels has been completed, as shown in FIGS. 11(a) and 11(b). This optical imaging probe is made of stainless steel, having a long and thin arm that better matches the dimension requirement for laparoscopic surgery. The 7 channels consist of 3 bifurcated channels, which serve for both light delivery and detection, and 4 non-bifurcated channels for just light detection, as shown in FIG. 11(c).

### The Probe and Imaging System

Examination of the vis-to-NIR imaging system is achieved by simulating the characteristics of various tissues and their interfaces in an in vitro laboratory model, also known as phantom.

Spatial Resolution Studied with a Single Source-Detector Pair Probe

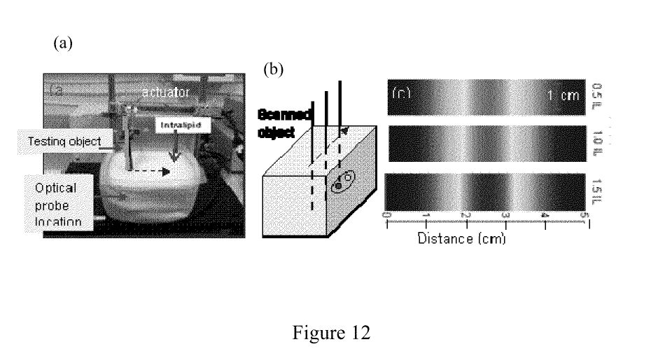

FIG. 12(a) depicts a phantom constructed having an object with high absorption surrounded by intralipid (IL) solution. This demonstration determines the feasibility of imaging objects contained beneath a fatty layer as is the case with bile ducts in the porta hepatis. Scanning a single source-detector pair probe across the fatty layer surface is equivalent to scanning the hidden object across the probe, as demonstrated in FIG. 12(b), while the optical reflectance is taken. FIG. 12(c) shows reflectance images for the hidden object, 4 mm below the probe, with IL concentrations of 0.5%, 1.0%, and 1.5% (to represent a different degree of fatness). The optical reflectance signals were recorded as the hidden object was scanned across the probe in steps of 1 mm. The data shown were taken from the 9.5 mm-separated pair of transmission-reflection probe fibers. Scanning reflectance readings were repeated while stepping the probe at different vertical positions, thus assembling a two-dimensional image of the heterogeneity on the surface of the

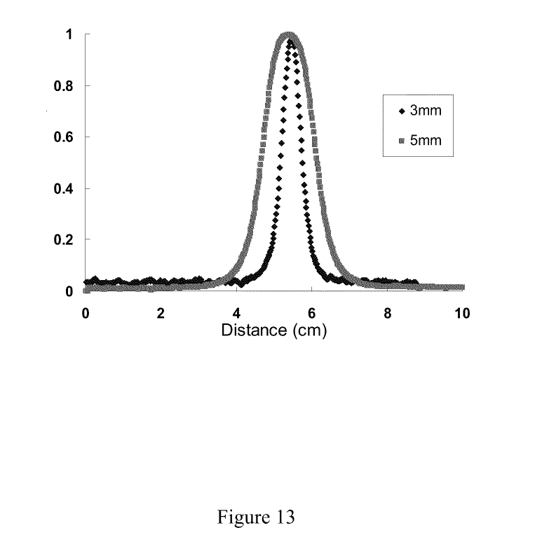

The next step is to decrease the source-detector, S-D, separation to improve the spatial resolution. FIG. 13 shows the reflectance data taken for the object with a 3-mm (thinner curve) and 5-mm (wider curve) diameter, respectively, with a 3-mm source-detector separation. The figure shows that a shorter separation allows for better spatial resolution if the object is superficial. By using this technique, it greatly improved spatial resolution and obtain nearly perfect match between the expected and calculated sizes for the embedded object.

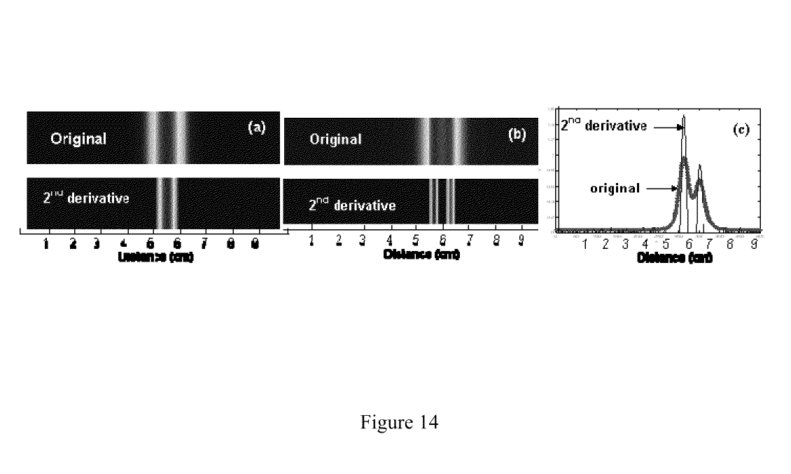

An example of the improved spatial resolution by operating the spatial second derivative on the measured signal profiles is shown in FIG. 14(a), where the object was 4-mm deep. More importantly, the spatial 2^{nd} derivative technique can significantly improve spatial resolution for multiple hidden objects. FIG. 14(b) demonstrates a good comparison between the original measured reflectance and the processed image profile taken from a tissue phantom containing two 3 mm-diameter absorbing objects, separated by 6 mm center-to-center apart. FIG. 14(c) is a 2-D profile plot for the same data, demonstrating that the 2^{nd} derivative process remarkably improves (or narrows) spatial resolution. However, these images do not provide depth resolution. A more mathematically rigorous approach for image reconstruction to achieve better spatial resolution and faster computational speed were developed

Spatial Resolution Studied with the Linear-Array Imaging Probe

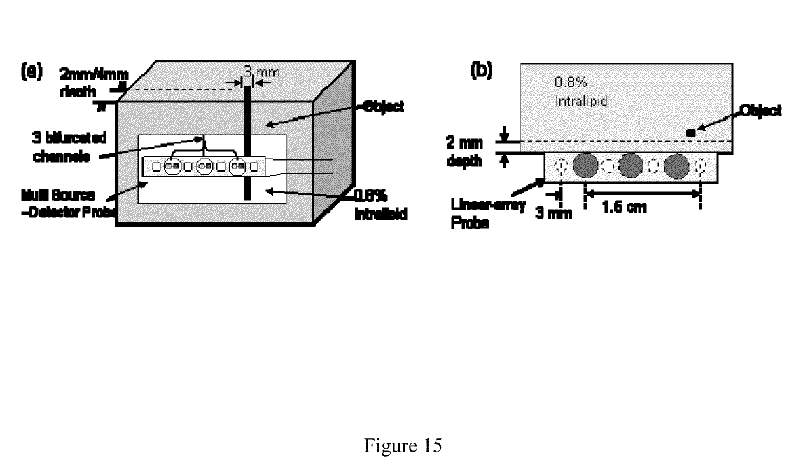

The present invention demonstrated how absorbing heterogeneities perturb the optical reflectance signals based on Monte Carlo computer simulations; the demonstration include reflectance data in the 3.5-14 mm source-detector separation range. With the recently implemented, linear-array imaging probe (FIG. 11), phantom model was used to image a hidden absorbing object using the linear-array probe without scanning the object (The setup and the probe location used in the phantom are shown in FIG. 15. The light sources through the bifurcated channels (3 red circles in FIG. 15(b)) were switched on sequentially while all the 7 detector channels recorded the reflectance data.

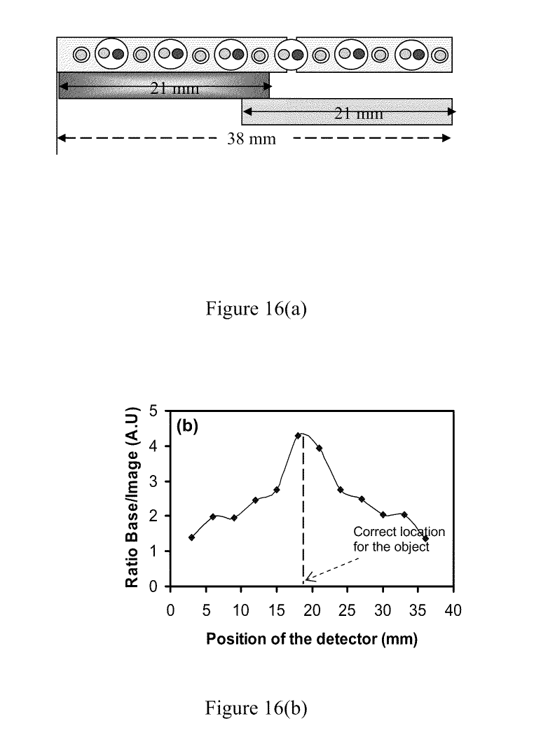

With such a phantom setup, the optical reflectance taken from the multiple channels provided spatial profiles similar to FIG. 15; the spatial resolution for the object near the edge seemed to be low due to the limited source-detector pairs near the length of 1.8 cm (see FIG. 15 (b)). To improve the spatial resolution near the edge, the probe array dimension was increased from 1.8 cm to about 4 cm by taking two consecutive sets of reflectance readings by scanning the array probe in two contiguous locations (FIG. 16(a)), and then combining the two sets of data together to form the spatial profile of the reflectance. With such an extended linear-array, a position-dependent, optical reflectance perturbation profile was plotted (FIG. 16(b)), which shows the hidden absorption object accurately.

### Diffuse Photon Propagation Models for the Sub-Surface Localization of Absorbing Heterogeneities

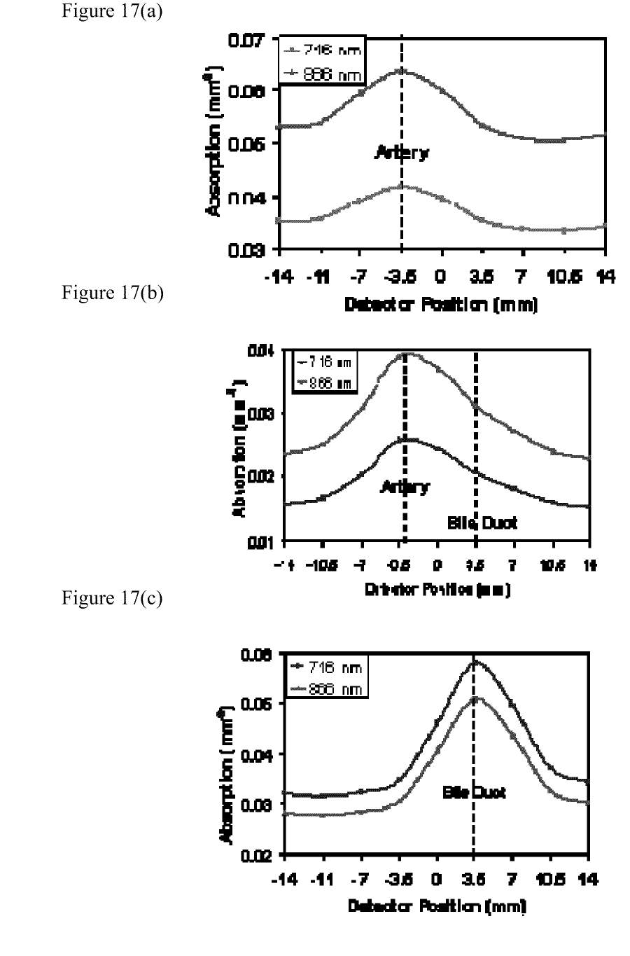

The present invention employed a two-step approach for localizing absorbing heterogeneities; the first step localizes them on the fatty tissue surface and the second step estimates their depth in fat. The demonstration simulated by Monte Carlo spectrally-resolved reflectance measurement scenarios where a 2 mm diameter artery is centered at 4 mm below the surface (FIG. 9(c)) and one where the same artery is 7 mm away from a 4 mm diameter bile duct (FIG. 9 (d)). The spectrally-resolved reflectance data were then fitted, resulting from stepping the source location in 3.5 mm intervals along the linear probe (emulating the measurement geometry of the actual intraoperative probe shown in FIG. 10), to a semi-infinite medium diffuse photon propagation model. This model produces an estimate for an average absorption and scattering coefficient within the banana-shaped probe tissue volume, which translates laterally as the source is moved. As the source location is stepped (or scanned through the fiber channels in the real demonstrations) from left to right (FIG. 9(c)), the resulting reflectance fit to the semi-infinite medium model exhibits greatest change in the wavelength-dependent absorption coefficients for locations where the banana-shaped photon density overlaps the true artery position (FIG. 17(a)). This data analysis approach accurately localize two absorption heterogeneities simultaneously (as in FIG. 9(d)) and correctly identify which one was the artery and which one was the bile duct. The fitted absorption coefficient along the surface of tissue peaked near the true artery location and was much lower at the bile duct location (FIG. 17(b)). Conversely, the bile absorption coefficient was highest at the center of the true bile duct's location (FIG. 17(c)). The difference between arterial blood and bile in FIGS. 17(b) and 17(c) can also be seen by the difference in the relative absorption amplitude at 716 nm (where bile is more absorbing) versus at 866 nm (where arterial blood is more absorbing) as expected from their known absorbance spectra.

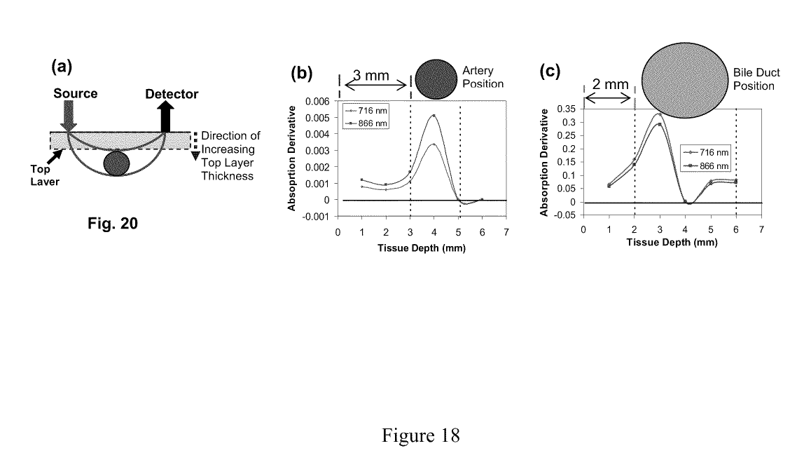

In the second step, the same spectrally-resolved reflectance data to a two-layer model of diffuse photon propagation were fitted and use the top layer thickness as a depth gauge for the absorbing heterogeneity. The logic of this approach is outlined in FIG. 18(a): The top layer partitions the banana-shaped probe volume in two (the part covered by the light blue area and the one that is not). If the top layer thickness is shallow, the part of the “banana” overlapping the top layer does not sample the absorption heterogeneity (as in FIG. 18 (a)), and the resulting fitted absorption coefficient for the top layer is similar to that of the background fatty tissue. As the top layer thickness is made incrementally larger, the portion of the ‘banana’ overlapping the top layer eventually cover part of the absorbing heterogeneity, which results in an jump for the fitted top layer absorption coefficient. The top layer thickness value where the absorption coefficient transitions from a low to a high value, as quantified by its depth-wise derivative, provides a reasonably accurate depth estimate for the location of the top side of the artery (FIG. 18(b), 3 mm below the tissue surface) or of the bile duct (FIG. 18 20(c), 2 mm below the tissue surface). Knowledge of the depth of a vessel is very useful to surgeons as it informs them how deep to cut without damaging that vessel. As the top layer thickness is increased further in the calculation for the apparent absorption derivative, it eventually cross the lower boundary of the artery or of the bile duct. The transition from the highly absorbing artery or bile duct back into the low absorbing fatty tissue produces a decreased or negative absorption coefficient derivative, as seen in both FIGS. 18 (b) and (c).

### Linearized Image Reconstruction for the Generation of Two-Dimensional, Depth-Resolved Images of Absorbing Heterogeneities

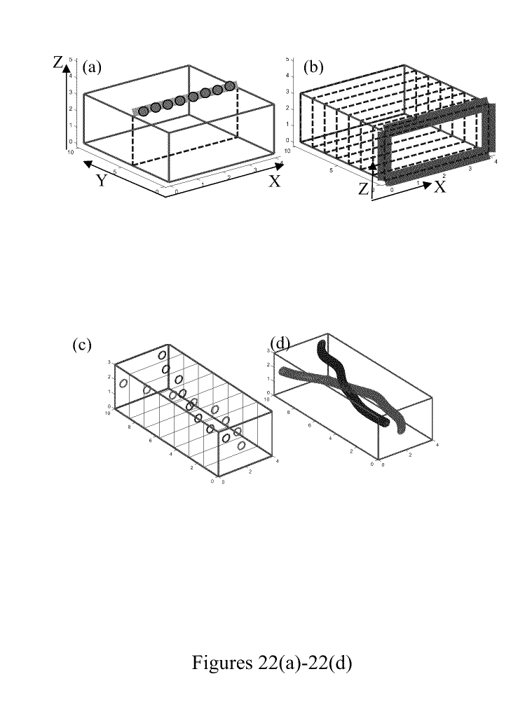

The present invention demonstrates feasibility of producing depth-resolved images of biliary tree tissue slices lying directly underneath the linear-array probe (shown in FIG. 10 or 11). Due to intraoperative spatial constraints, the linear-array probe can be placed in a near-perpendicular direction relative to the hepatic artery and the CBD. Therefore, the reconstructed vis-to-NIR reflectance images are similar to the ultrasound, B-scan mode and produce cross-sectional views of these vessels embedded in fat (see FIG. 22). To demonstrate the feasibility for this approach, a computationally efficient image reconstruction algorithm was employed, based on the perturbation solution of the diffusion equation that has been previously used for face-on imaging. This algorithm is also adapted to the B-scan geometry.

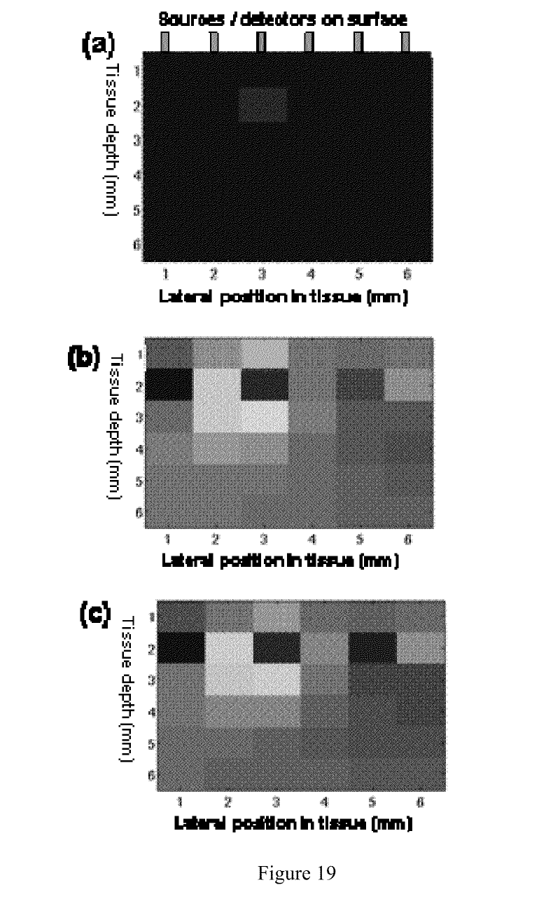

Synthetic data simulating the detector response based on the perturbation solution of the diffusion equation were generated. For an absorption perturbation map where most pixels had the absorption coefficient of fat and one pixel had that of arterial blood (FIG. 19(a)), the image reconstruction performed well in recovering the highly absorbing pixel location (FIG. 19(b)). In fact, an equally accurate absorption heterogeneity map was also retrieved (FIG. 19(c)) with a significantly different scattering coefficient for fat, relative to the one used to generate the measurements, i.e., fat scattering used for FIG. 19(c) was 30% below the actual baseline. The results shows confidence that accurate knowledge of the background tissue optical properties is not essential to recovering reasonably accurate absorption maps.

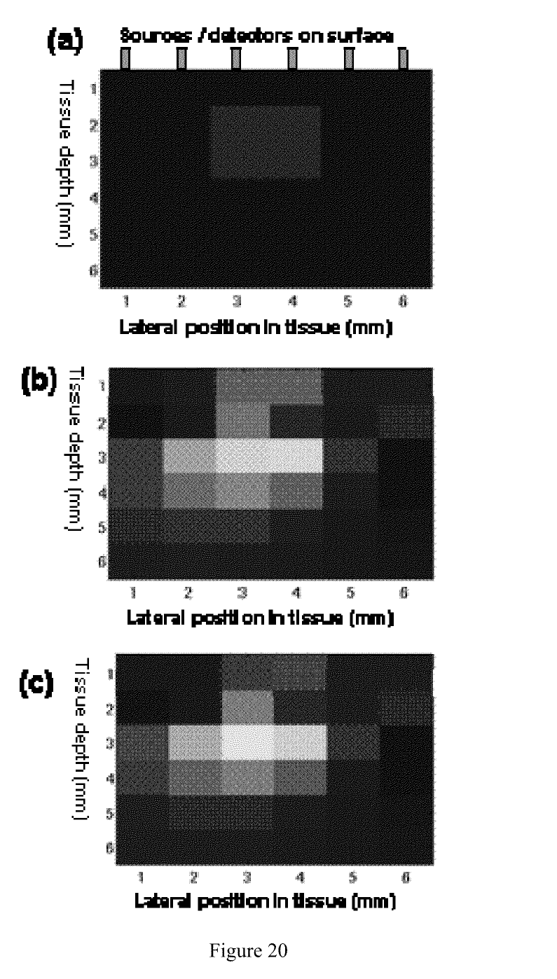

The present invention also demonstrated the capacity of the reconstruction algorithm to reconstruct a 2×2 pixel absorption heterogeneity (FIG. 20(a)) when the scattering coefficient of background fat accurately are known (FIG. 20(b)) or not known (FIG. 20(c)). Interestingly, the reconstructed heterogeneity map appears to be giving more weight to the pixels closer to the tissue surface. This has also been observed for the more time-consuming iterative reconstruction. This surface-weighted behavior in an emission tomography setting when reconstructing images from measurements at a single wavelength have been observed in the past. The present invention uses multi-wavelength reflectance data to alleviate it.

It is important to note that one of the goal of the present invention is not only to retrieve accurate tissue optical property maps, but ones where differences in the relative pixel values highlight the true position of underlying vessels and ducts (common bile duct, CBD, and cystic duct) in order to guide the surgeon during laparoscopic cholecystectomy. The present invention shows the capacity of our algorithm to reconstruct absorption heterogeneities using Monte Carlo simulated, spectrally-resolved reflectance data. The present invention also simulate reflectance data for the case where both the hepatic artery and the CBD are in the field of view. In this case B-scan reconstructions can discriminate between these two structures as was achieved in alternative vessel localization approach were demonstrated.

Reconstruction of Three-Dimensional Image Volumes from Two-Dimensional Image Sets

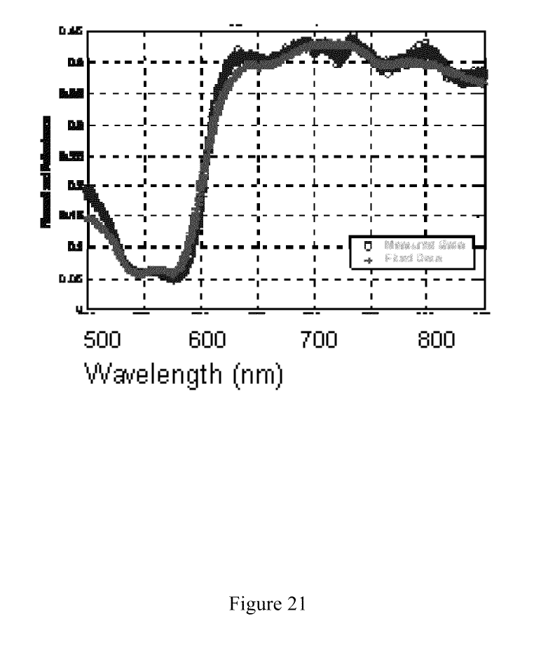

Determination Of Baseline Optical Properties For Intraoperative Tissues. In order to reconstruct the biliary tree structure images accurately, it is important to have a priori information regarding the optical properties of the tissues being imaged. By reference to portal structures, showing locations of the common bile duct and hepatic duct, portal vein and hepatic artery, a skilled artisan can see the major light scatterer is portal fat, and the main absorbers are venous and arterial blood as well as bile. These optical properties by analysis of the spectrally-resolved reflectance data with a non-diffuse empirical photon migration model, which was recently developed by Zonios and Dimou were determined. Each bifurcated source fiber can be used along with one of the adjacent detector fibers (see FIG. 10, 11, 15, or 16) to obtain short source-detector reflectance readings so that only a small and superficial volume of tissue is probed. FIG. 21 shows a comparison between data that was obtained from a short distance reflectance measurement on ex vivo porcine fat and the fitted data based on that model. In a similar fashion, the optical properties of human portal fat and bile samples obtained during surgery were determined. Such fitting takes less than 1 minute to obtain the results, so it is very feasible for real-time performance during surgery.

Reconstruction of Three-Dimensional Image Volumes from Two-Dimensional Image Sets

To demonstrate the feasibility of creating three-dimensional image volumes from two-dimensional image reconstructions of sequential image planes, current image reconstruction algorithm with computer-simulated optical reflectance data to reconstruct the blood vessel and biliary tree locations in the measurement domain (FIG. 22(a)) were used. It should be noted that this algorithm is based on the iterative solution of the photon diffusion equation on a finite element mesh. The image reconstruction domain was 4 cm by 3 cm, and the vessel diameters were all 0.5 cm. The green bar in FIG. 22(a) represents the linear-array sensors' location with a 4-cm length and with 8 bifurcated, source-detector pairs (magenta dots). The measurements are made at N=8 discrete locations on the surface (FIG. 22(b)). The simulated optical measurements (at a NIR wavelength) were used to reconstruct the location of the blood vessel and biliary tree structures in each section (FIG. 22(c)). A smoothed image map was obtained by interpolation of the discrete locations where the vessels or bile duct were reconstructed in each two-dimensional plane (FIG. 22(d)). These findings prove the feasibility of creating three-dimensional image volumes from two-dimensional image sets.

In summary, the present invention is a vis-to-NIR imaging probe system integrating the use of image-reconstruction algorithms to visualize the common bile duct during laparoscopic cholecystectomy.

In practice, the probe system functions similarly to an ultrasound device, enabling the operator to scan the tissue and map the structures located beneath the surface. In this way, a surgeon could trace the cystic duct back from the gallbladder to the common bile duct. The anatomical relationship between the cystic duct and common duct bile and hepatic ducts can be established prior to gastroduodenal ligament dissection. Because of the fundamentally differing properties between ultrasound and light imaging, it is anticipated that a probe developed based on reflected spectroscopic technologies results in bile duct imaging that is very high resolution and more reliable than currently available ultrasound devices.

### Instrumentation Details

The present invention demonstrates an intraoperative imaging device facilitating a surgeon's ability to identify biliary structures. It also demonstrated that a vis-to-NIR probe built which can discern biliary structures and that this technology provides a feasible solution to the minimization of iatrogenic bile duct injury during cholecystectomy.

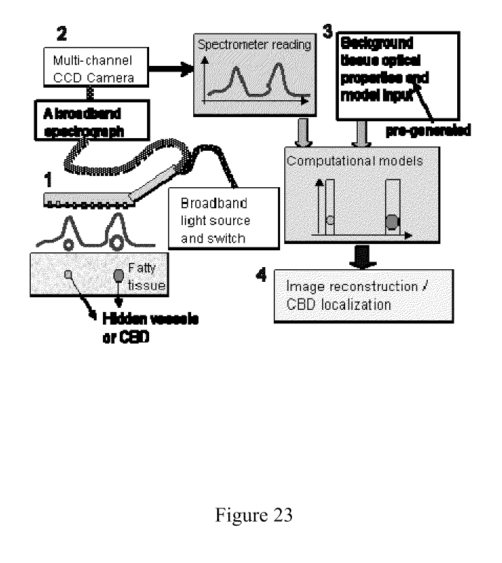

FIG. 23 shows the overall vis-to-NIR imaging system design: (1) intra-operative probe, (2) CCD-based spectroscopic imager, (3) computational models to improve image resolution at near real-time processing speeds, and (4) classification algorithms for the identification of porta hepatis structures.

### Design and Build an Intra-Operative Vis-to-NIR Probe

As the present invention demonstrated earlier, the imaging depth is reliant upon the source-detector separation, and the spatial resolution depends on the number of source-detector pairs. So, a sufficient lateral length and an adequate number of source-detector pairs are needed.

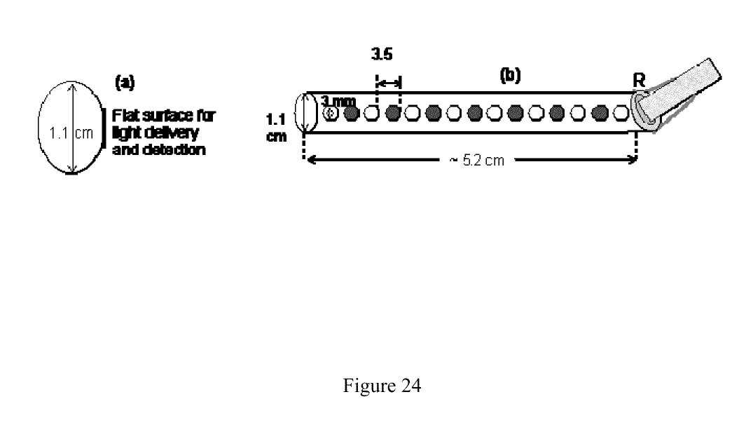

The present inventors designed an oval-shaped probe having an outside diameter of 1.1 cm (FIG. 24(a)) to accommodate the 1-cm port size that is the current standard for laparoscopic surgery. The probe design is shown in FIG. 24. The circles in FIG. 24(b) represent the light delivery and detection channels connected to multi-fiber optical bundles. Red circles denote bifurcated fiber bundles connected to a time-shared light switch. When light is delivered to a fiber labeled as a red circle in the schematic, the adjacent 3 channels (3.5 mm to 10.5 mm away from the light emitting fiber) read the optical spectra. “Red-circle” channels need to both deliver and detect light, necessitating bifurcated fibers. This probe traverse a ˜5.2 cm linear distance resulting in 14 sets of 3.5-mm-separation readings, 12 sets of 7-mm-separation readings, and 12 sets of 10.5-mm-separation readings. This linear array provides sufficient flexibility in fiber selection to optimize image formation and tissue penetration of the vis-to-NIR light. The joint “R” in FIG. 24(b) was designed to be compatible with laparoscopic surgery in much the same way current intraoperative ultrasound probes are constructed. The length of arm L is approximately 30-40 cm.

### Design, Implement, and Calibrate a Vis-to-NIR Imaging System

System Design and Implementation. The present inventors built a multi-channel, vis-to-NIR imaging system using a spectrograph and a CCD camera. This enables simultaneous spectroscopic recording from various locations over the porta hepatis (FIG. 18b). The design for the proposed CCD-camera-based, spectroscopic imager is a modification of existing multi-channel tomographic imager used for tumor imaging. FIG. 17 depicts the existing 8-channel, broadband, NIR imager (a) with eight bifurcated fiber bundles (b, c). FIG. 17(c) shows a static tissue phantom with a blood-filled test tube embedded in lipid.

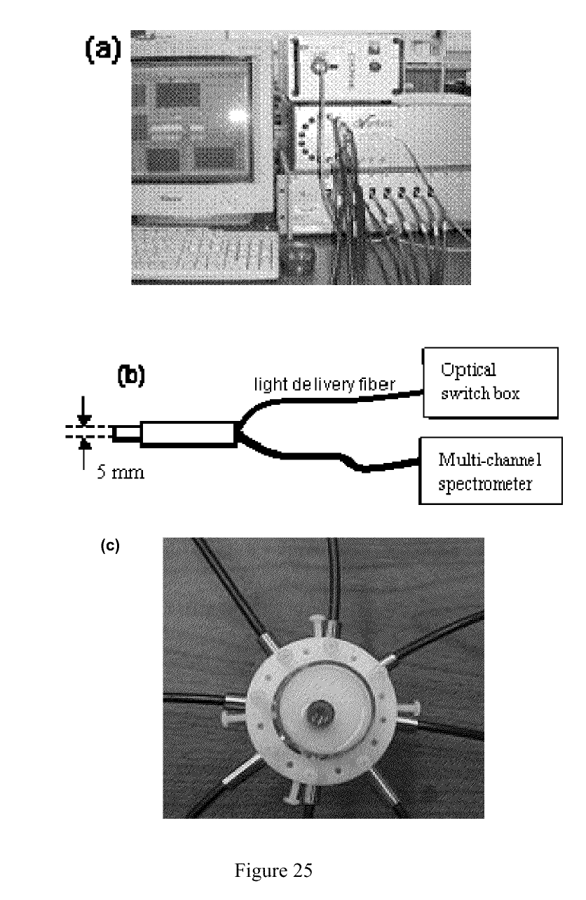

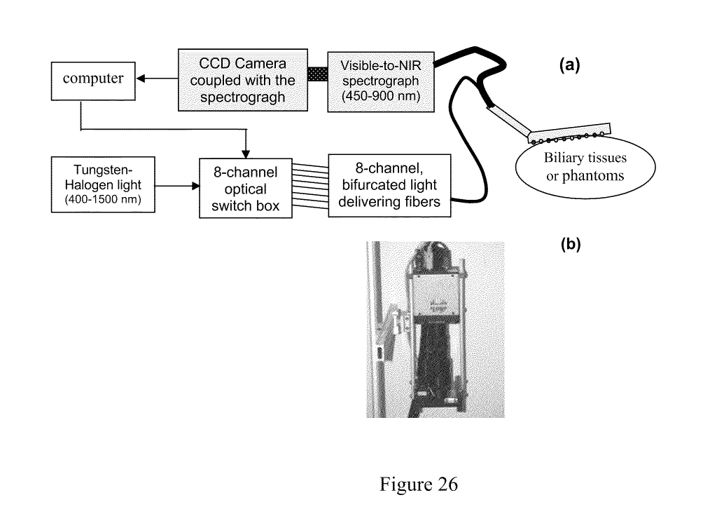

For applications, the present invention replaces the 8-channel spectrometer with a spectrograph and CCD camera. This facilitates more rapid data acquisition from more channels than was previously possible. The schematic diagram for the new design is shown in FIG. 26. The present invention needs about 7 bifurcated (“red circles” in FIG. 25) and about 8 non-bifurcated fiber bundles for the new probe; 15 fiber bundles enter the visible-to-NIR spectrograph (450-900 nm), and the 7 bifurcated fiber branches are connected to the multi-channel optical switch.

The present invention arranges multiple fiber bundles in a linear array. This set the array in the imaging spectrograph's focal plane of the CCD camera. An optical focusing system at the spectrograph slit was added. The fiber bundles are imaged through a high-dispersion grating in the spectrograph by a 496×656, 12-bit CCD camera. FIG. 26(b) shows an existing CCD camera that has been for imaging of tumors and can be used for the proposed project. Signals acquired by the CCD includes both spectral (recorded in rows of the 2D CCD array) and spatial (in columns of the CCD array) information. The grating dispersion and spectrograph relative flat field are selected for single acquisition of a 250-nm-width spectrum.

### System Calibration

The CCD images are calibrated to remove interference derived from the light source, spectrograph, fibers, and the CCD camera. Measured optical spectral intensity, Rₜᵢₛₛᵤₑ(λ, x, y), at a discrete locations (x, y) should be background subtracted and normalized to a standard calibration sample:

$${R}_{cal} ( λ , x , y ) = \frac{\begin{matrix}{R}_{tissue} ( λ , x , y ) - \\ {R}_{back \_ tissue} ( λ , x , y )\end{matrix}}{\begin{matrix}{R}_{standard} ( λ , x , y ) - \\ {R}_{back \_ standard} ( λ , x , y )\end{matrix}} , \qquad Equation ( 2 )$$

R_{back}_{_{—}}ₜᵢₛₛᵤₑ(λ, x, y) and R_{back}_{_{—}}_{standard}(λ, x, y) are the measured background spectral intensities (with the light source off) from both the tissue and standard sample, respectively. R_{standard}(λ, x, y) is the spectral intensity from the standard sample at location (x, y). A standard sample is available in our lab with a high reflectivity of >99.9% and a flat spectral band at 500-900 nm. After the calibration is performed, R_{cal} (λ, x, y) can be used for subsequent imaging processing and reconstruction.

### Computational Methods for the Localization of Biliary Tree Structures

Light scattering by fatty tissues hinders surgeons from visualizing biliary tree structures directly. At NIR wavelengths light can penetrate tissues deeper, but it nevertheless experiences strong scattering. The result of NIR photons being scattered multiple times is degradation of spatial resolution and reduction of contrast in reflected light images. Therefore, use of NIR sensitive cameras, in lieu of the human eye, to view NIR reflected light images may still not produce enough resolution and contrast to reliably localize biliary tree structures intraoperatively. To solve this low spatial resolution problem, the present invention demonstrates two novel computational approaches to rapidly localize these structures at the portal fat surface and also to estimate their depth within that tissue: (1) a two-layer diffusion model using the interface between top and bottom layers as a depth localization tool for absorbing heterogeneities, and (2) a linearized image reconstruction algorithm to obtain ultrasound-like, two-dimensional images.

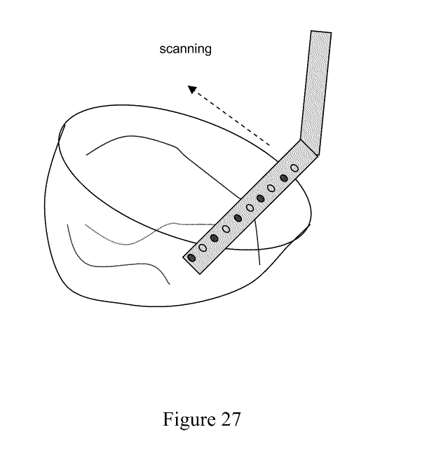

Since multi-separation probe is a linear array, it is needed to move the probe laterally over the tissue surface (FIG. 27) in order to build up three-dimensional images from sequential two-dimensional ones (as was done in FIG. 22). In approach (1), as just mentioned above, the image volume data represent the top layer absorption coefficient for different top layer thicknesses, whereas in approach (2) they represent an interpolated stack of sequential two-dimensional maps of the change in absorption coefficient over that of baseline fat absorption. It is important to point out that both of these computational approaches can be completed within a few seconds, or less, by a state-of-the-art personal computer. Therefore a long-term, software-hardware implementation where surgeons can look at three-dimensional volumes being created in real time, slice-by-slice, on a computer screen in front of them as they acquire data intraoperatively was envisaged.

### Two-Layer Diffusion Model and Bulk Intraoperative Tissue Optical Property Estimation

The intraoperative volume geometry to be imaged consists of the biliary tree, the portal vein and the hepatic artery embedded within a depth of 2-6 mm in fatty tissue. Multi-spectral reflectance data from that tissue geometry to a two-layer model of diffuse photon propagation was fitted. The idea is that a two-layer model enables fitting of two distinct absorption coefficient values, one for the top layer and one for the bottom one. The value of these two absorption coefficients strongly depend on the depth of the layer interface relative to that of absorbing heterogeneities, such as the hepatic artery or the CBD. If that interface is located above these absorbing heterogeneities, then the top layer diffusion coefficient are similar to that of portal fat and therefore have a low value. If the depth of the layer interface is continually incremented (FIG. 18(a)), it eventually crosses the depth where the hepatic artery or the CBD are located. Once these structures are found in the top layer of the two-layer geometry, the top layer absorption coefficient naturally increases (FIGS. 18(b), 18(c)). The interface depth where that increase in top layer absorption occurs in the fitted data are used as an estimate for the depth of these structures. In the case where the artery and CBD are in close proximity and the banana-shaped probe distribution crosses both, their depths from the tissue surface can still be estimated independently, FIGS. 17(b) and 17(c). This is enabled by the fact that our two-layer diffusion model fits multi-spectral reflectance data and outputs spectrally-resolved absorption coefficients that are expressed as the sum of contributions from chromophores of known absorbance profiles, such as blood and bile. Therefore, the depth where an increase in top layer absorption occurs can be determined independently for each distinct chromophore.

It should be noted that a two-layer diffusion model can have up to five fitting parameters: the absorption and transport scattering coefficients of the top and bottom layers as well as the top layer thickness. It has previously been shown that this is an ill-posed parameter estimation problem. More robust estimation of the absorption coefficients of the two layers can be attained if prior information is included into the model so that the number of fitting parameters can be reduced. This particular clinical application lends itself to such a simplification as the dominant background tissue is the rather homogeneous and low-absorbing portal fat. Therefore, if the portal fat optical properties can be known a priori along with the wavelength-dependent absorbance of blood and bile, the fitted absorption coefficients, representative of the tissue volumes sampled by the corresponding banana-shaped probe volumes, can be estimated with greater confidence. The bulk optical properties of human portal fat are determined by application of the methodology described. Multiple measurements were performed on fat samples from multiple patients, and population-averaged optical property values were deduced and applied to all subsequent data processing.

### Linearized Diffusion Image Reconstruction Algorithm for Intraoperative Biliary Tissues

There are very few image reconstruction demonstrations performed in B-scan geometry to date, and these employ a time-consuming iterative solution of the diffusion equation. When real-time results are needed, as is the case for intraoperative measurements, investigators use computationally efficient algorithms that typically employ a perturbation solution of the diffusion equation. The perturbation solutions do not retrieve quantitative results for the tissue optical properties, but they can produce maps of absorption heterogeneity with great sensitivity. This approach has been adapted to reconstruct images in the B-scan geometry.

A linearized perturbation diffusion methodology employing the Rytov approximation along with a regularized inversion technique are employed for reconstructing two-dimensional images from CW (continuous wave) spectrally resolved reflectance data. According to this approach, the set of measurements y representing the signal change over baseline signal, originating from vis-to-NIR light absorption by portal fat in this case, can be linked to the spatial map of blood or bile absorption changes x by a detection sensitivity matrix A through the relation y=Ax. The matrix A weighs appropriately the contributions to expected signal change in each detector y for different activation locations x, depending on their distance from the corresponding detectors. This linear relation between y and x can in principle be solved to yield a depth-resolved two-dimensional map of blood and bile absorption changes x=A⁻¹y, over that of baseline fat. However, this inverse problem is ill-posed, meaning that there are many estimated absorption maps that yield similar detector signal sets. A robust solution to this inverse problem is given by a method known as the Moore-Penrose generalized inverse:

x=A^{T}(AA^{T}+αsₘₐₓI)⁻¹y Equation (3)

where I is the identity matrix, sₘₐₓ is the maximum eigenvalue of AA^{T}, and α is the regularization parameter that is empirically set to be 10⁻³.

It should also be noted that although the reconstructions are two-dimensional, the algorithm computing vis-to-NIR photon propagation in tissues are three-dimensional. As the reconstructed optical property images contain spectrally resolved information, it can calculate the relative contributions from known chromophore spectra in each image pixel. This enables the classification of those pixels as bile, arterial or venus blood, or fat. Population-averaged optical properties are assumed for portal fat.

Radial Basis Function Modeling of Spectra Obtained from the Animals

After obtaining sets of animal spectra from the multi-separation, linear-array probe and removing the broadening effects of fat, a 2D image that images an otherwise hidden bile duct, portal vein or hepatic artery was formed. Three derived parameters A, B, and C to characterize different tissue types were used. The observations showed that the vis-to-NIR spectra observed for biliary tissue are very different from adjacent vascular structures. The RBF (radial basis function) parameters were consistent from one animal to the other. However, to ensure stability of these estimates more animal measurements must be obtained. Vis-to-NIR measurements from 18 more pigs to ensure statistical validity and consistency of the measured parameters were obtained. The set of median values obtained from the basis function parameters (A, B, C) are used as the identifiers for each tissue. These parameters are used for the classification purposes.

### Tissue Classification: Minimal Distance Method and Supporting Vectors Machine

Minimal Distance Method (MDM). There are two phases in tissue identification using MDM: the training and classification phase. (1) Compute the center locations of each biliary tree structures in the 3D A-B-C space; this computation is based on the derived parameters from the data taken either from laboratory phantom or animal models, (2) calculate the distances from all other data points to the center for each biliary tree structure, and plot all other points in the A-B-C space, and (3) compute the standard deviation, σ(i), for the distances from all data points to the ith center for each of biliary tree structures.

In the first phase, (1) the sets of A, B, C parameters to be identified were obtained, (2) the distances, R(i), to the i^{th} center of each biliary tree structure (i=1, 2, . . . 5) were calculated, (3) the normalized distance as R_{N}(i)=R(i)/G(i) between the unknown data point and each of the i^{th} center were computed, and (4) the minimal normalized distance, R_{N}(i)_min, which corresponds to the biliary tissue type. R_{N}(i) was calculated and has the feature of the Mahalanobis distance and the classification is based on the determination of minimal Mahalanobis distance. This methodology was used to analyze and classify the vis-to-NIR data taken from laboratory phantoms, animal models, and human surgery.

SUPPORT VECTORS MACHINE (SVM): SVM was originally designed for binary classification. For multiple parameters, the data points may be utilized in a n-dimensional space. In SVM, the classification is accomplished by establishing separation of hyper-surfaces that separate the two sets of data. The maximum margin separating hyper-surfaces are found by solving an optimization problem, and the data points (the vectors in n-dimensional space) that are closest to this separating surface are known as the support vectors.

The present invention uses a “one-against-one method” for tissue classification. To classify the 5 biliary tree structures (hepatic artery, portal vein, CBD, cystic duct, and gallbladder), it is needed to train 10 SVMs (combinations of 5 biliary tree structures taken two at a time). Once the SVMs are trained in the classification application, a “max wins” voting strategy is used to classify the biliary tissue types. With this strategy, each SVM classifier vote for one of the biliary tissues as the possible tissue type. The structure that gets the most votes are selected as the identified biliary tissue type. Similarly to the MDM, SVM area used in data analysis for all the laboratory phantom, animal, and human demonstrations.

### Efficacy of the Imaging Device and Classification Algorithms in Animals

Once optimized in vitro, the probes are tested for their efficacy in identifying bile ducts in animal models of open and laparoscopic surgery. Further refinements in the probe and its analytic software were made prior to testing the device in humans.

Pigs were chosen for animal models. This choice was made because pigs have a reasonably large porta hepatis with common bile duct, hepatic artery and portal vein of sufficient size that our imaging techniques should be able to discern them. Approximately 18 pigs were used: 6 for open surgery, 6 for laparoscopic surgery, and 6 for testing the effects of respiratory interventions to enhance imaging contrast.

Pigs were anesthetized. Following positioning on the animal operating room table, the abdomens are opened and the liver retracted as is done in open surgery. The porta was exposed such that the optical scans described in the proposal can be obtained. The optical spectra were digitally stored for subsequent analysis. For laparoscopic surgery, the liver was retracted as is done for these operations and the probe placed against the relevant structures following its introduction into the abdomen via a 1 cm port. For every organ, multiple sets of measurements were obtained by moving the probe onto different locations on the porta or gallbladder. During some of the demonstrations, inhaled gases for the animal may be altered from regular air with 21% oxygen to different oxygen percentages, ranging from 15% to 100%. This varies tissue and blood oxygenation that could affect optical images. These effects, as well as their use as a possible “contrast” agent were observed.

### Human Common Bile Duct Imaging

The present inventors conduct human measurements in the operating room using the newly developed vis-to-NIR imaging system, the laparoscopic linear-array probe, image reconstruction algorithm, and the classification algorithms to demonstrate the ability of the optical imaging technique for identification of the common bile duct during human surgery.

The newly constructed and porcine validated probe were used during human gallbladder operations (n=20 in total, n=12 for open surgery and n=8 for laparoscopic surgery). During open surgery, the probe was rested on the gallbladder, liver and any readily available mesenteric blood vessel. Vis-to-NIR spectra were recorded and stored for later analysis. Fluoroscopic cholangiography were performed (Patients selected were those for whom cholangiography is planned). The optical probe was placed over the bile duct whose location were determined by the relationship between external landmarks and the bile duct location observed on a cholangiogram. The hepatic artery was located by digital palpation and the probe placed over it. Optical readings were repeated 5 times for each structure.

The optical spectra were subjected to the image reconstruction and classification program. Success was defined as having a greater than about 95% rate of correct tissue identification. If initial attempts to correctly identify the various porta hepatis structures were not obtained, the observed vis-to-NIR spectra were subjected to re-analysis by the mathematical modeling algorithms to develop a set of radial basis functions and classification scheme specific for human tissues. The present inventors use the data taken from 6 human subjects to develop/confirm the classification algorithm. Following this adjustment, the probe was tested on another 6 human patients. Multiple readings were obtained (n=5) from each human biliary structure and tested to determine reproducibility.

In addition to demonstrations performed on patients undergoing open operations (n=6), the probe in laparoscopic cases (n=8) was evaluated. Because identification of the bile duct and vascular structures is less than for open operations, these demonstrations concentrated on examination of the gallbladder and any other large structures that can be definitively identified and the probe placed on it. The next major step was to show an imaging system that incorporates a software/hardware solution for the algorithms derive. An imaging system was built so that the probe's output can be transplanted to a screen just as an ultrasound image is displayed.

### Statistical Methods

A student-t test was utilized to determine those parameters that are significantly altered between CBD and cystic duct and other anatomical structure such as blood vessels and fat. Cross-correlations were examined to determine causal, complementary, parallel, or reciprocal relationship, especially a structural, functional, or qualitative correspondence between the imaging modalities and surrogate markers within each modality. Furthermore, ANOVA (Analysis of Variance) was applied to conduct comparisons among the modalities and surrogate markers within each modality.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An apparatus to visualizes one or more tissues in vivo comprising:
an electromagnetic radiation source capable of producing a continuous wave broadband light;
at least one optical probe connected to the electromagnetic radiation source;
a multi-channel CCD array spectrometer detector connected to the optical probe capable of collecting near infrared wavelength emissions;
at least one computer connected to the multi-channel CCD array spectrometer detector, wherein the computer comprises one or more image evaluation algorithms; and
at least one display connected to the computer to generate images from the one or more tissues and display results from the image evaluation algorithms.

2. The apparatus of claim 1, wherein the optical probe comprises a fiber optical bundle having one or more non-bifurcated channels for light delivery fibers and one or more bifurcated channels to collect the near infrared wavelength emissions.

3. The apparatus of claim 1, wherein the near infrared wavelengths emissions comprise light between about 550 nm and about 900 nm.

4. The apparatus of claim 1, wherein the one or more tissues comprise a biliary tree.

5. The apparatus of claim 1, wherein the one or more image evaluation algorithms comprise a Radial Basis Function algorithm to facilitate the characterization of the one or more tissue based on observed reflectance spectra and to distinguish between tissue structures.

6. The apparatus of claim 1, wherein the one or more image evaluation algorithms further comprise a Minimal Distance Method algorithm to classify the one or more tissues.

7. The apparatus of claim 1, wherein the one or more image evaluation algorithms further comprise a two-layer diffusion model algorithm to localize heterogeneities, and a linearized image reconstruction algorithm to obtain ultrasound-like, two-dimensional images.

8. A method for imaging one or more tissues in a subject during a surgical operation comprising the steps of:
directing a continuous wave broadband light towards the one or more tissues using at least one optical probe;
collecting near infrared wavelength emissions from the one or more tissues using the optical probe;
analyzing the near infrared wavelength emissions using one or more image evaluation algorithms; and
reconstructing a two-dimensional or three-dimensional optical map of the one or more tissues based on results derived from the one or more image evaluation algorithms.

9. The method of claim 8, wherein the optical probe comprises a fiber optical bundle having one or more non-bifurcated channels for light delivery fibers and one or more bifurcated channels to collect the near infrared wavelengths emission.

10. The method of claim 8, wherein the near infrared wavelengths emissions comprise light between about 550 nm and about 900 nm.

11. The method of claim 8, wherein the one or more tissues comprise a biliary tree.

12. The method of claim 8, wherein the step of analyzing the near infrared wavelength emission further comprises characterizing the one or more tissue using a Radial Basis Function algorithm to facilitate tissue identification based on observed reflectance spectra and to distinguish between tissue structures.

13. The method of claim 12, wherein the step of characterizing the one or more tissue using a Radial Basis Function algorithm, further comprises classifying the one or more tissue using a Minimal Distance Method algorithm.

14. The method of claim 13, wherein the step of classifying the one or more tissue using Minimal Distance Method comprises calculating variables using the equation:
$$S ( λ ) = {\sum }_{i = 1}^{N} {a}_{i} {e}^{( \frac{- {( λ - {λ}_{i} )}^{2}}{2 {σ}_{i}^{2}} )}$$

15. The method of claim 8, wherein the one or more image evaluation algorithms comprise a two-layer diffusion model to localize heterogeneities, and a linearized image reconstruction algorithm to obtain ultrasound-like, two-dimensional images.

16. The method of claim 8, wherein the surgical operation is cholecystectomy.
